(19) United States
(12) Patent Application Publication
(10) Pub. No.: US 2024/0082833 A1
(43) Pub. Date: Mar. 14, 2024

(54) supported metal catalyst with synergistic sites, a preparation method therefor and an application thereof

(71) Applicant: Beijing University of Chemical Technology, Beijing (CN)

(72) Inventors: Yanan Liu, Beijing (CN); Dianqing Li, Beijing (CN); He Yu, Beijing (CN); Ning Li, Beijing (CN)

(21) Appl. No.: 18/452,032

(22) Filed: Aug. 18, 2023

(30) Foreign Application Priority Data
Sep. 13, 2022 (CN) 202211110171.0
Mar. 21, 2023 (CN) 202310282510.1

(51) Int. Cl. B01J 37/02; B01J 21/04; B01J 27/045; B01J 27/051; B01J 35/00; B01J 35/10; B01J 37/04; B01J 37/06; B01J 37/08; B01J 37/16; C07C 1/30; C07C 5/32; C07C 29/145

(52) U.S. Cl. CPC B01J 37/0201; B01J 21/04; B01J 27/045; B01J 27/051; B01J 27/0515; B01J 35/0066; B01J 35/1014; B01J 35/1019; B01J 35/1038; B01J 35/1042; B01J 35/1047; B01J 35/1061; B01J 37/0236; B01J 37/04; B01J 37/06; B01J 37/08; B01J 37/16; C07C 1/30; C07C 5/322; C07C 29/145; C07C 2521/02; C07C 2523/28; C07C 2523/72; C07C 2527/051

(57) ABSTRACT

The present invention provides a supported metal catalyst with synergistic sites, a preparation method therefor and an application thereof. The preparation method of this catalyst is to utilize the unsaturated cubane-like structure, M cation with catalytic activity is introduced into the cluster core unit. By using the vertex vacancy as the capturing center, and adjusting the impregnation temperature to maximize the loading of the cluster precursor, as well as depending on the electrostatic adsorption of the support and the confinement of the cluster structural unit, the number of S vacancies and the distance between S vacancies and Miso sites are effectively controlled through liquid phase reduction and atmosphere treatment at room temperature to obtain supported X3MSx/Al2O3 catalyst with Miso-Vs synergistic sites. The method of the present invention achieves the joint enhancement of the activity, product selectivity, and stability of unsaturated carbon oxygen bond selective hydrogenation, carbon chlorine bond selective hydrogenation dechlorination, and carbon hydrogen bond dehydrogenation reactions. This catalyst is mainly used in various catalytic reaction processes in the fields of petrochemical, fine chemical, environmental chemical, and other fields. It has outstanding catalytic performance, excellent activity, selectivity, and good recyclability, and is easy to recover and reuse.

### TECHNICAL FIELD

The present invention belongs to the fields of petrochemical engineering, fine chemical engineering and environmental chemical engineering, and relates in particular to a supported metal catalyst with synergistic sites and a preparation method therefor.

The catalyst is mainly used in selective hydrogenation of C═O bond, hydrodechlorination of C—Cl bond and dehydrogenation of C—H bond.

### BACKGROUND ART

Global targets of sustainable development in the energy and environmental fields have seen site-isolated catalysts receive widespread attention due to their maximal atom utilization and special catalytic behavior, which is associated with low coordination number, quantum confinement of metal atoms, and/or strong interactions with the catalyst support. Traditional fabrication strategies for site-isolated catalysts have been developed, which are highly dependent upon the initial adsorption/binding of a metal precursor with a support and the introduction of second metal as well as often require lower metal loading to be effective.

For instance, Zhang et al. explored the impact of different particle sizes in Pt/Al₂O₃ catalysts on the activity and selectivity of propane dehydrogenation (PDH) reaction in Size Dependence of Pt Catalysts for Propane Dehydrogenation: from Atomically Dispersed to Nanoparticles, ACS Catal. 2020, 10, 21, 12932-12942. It was found that the activity of propane dehydrogenation increased with the decrease of Pt particle size, while the selectivity showed a volcanic curve. When Pt particles dispersed at atomic level, Pt₁/Al₂O₃ catalyst showed the most preferred selectivity, but its activity (<25%) still needed to be further improved. To solve this problem, researchers found that the introduction of a second component can not only highly disperse the active metals, but also modify the electronic structure, and thus enhance simultaneously the catalytic activity and selectivity. In the literature Construction of a Unique Structure of Ru Sites in the RuP Structure for Propane Dehydrogenation, ACS Appl. Mater. Interfaces 2021, 13, 28, 33045-33055, Yang et al. introduced a proper amount of P into Ru-based catalysts to form Ru-based phosphide catalysts. Compared with monometallic Ru-based catalysts, strong electron interaction was produced between P and Ru, which can further improve the adsorption capacity of reactants while maintaining high selectivity, thus enhancing the catalytic activity. In the literature Fabrication of Supported Pd—Ir/Al₂O₃ Bimetallic Catalysts for 2-ethylanthraquinone Hydrogenation. AIChE J. 2017, 63, 3955, Hong et al. further optimized the bimetallic structure, and obtained a series of Pd—Ir catalysts with alloy structure. It was found that the ordering degree of alloy had a significant influence on the selective hydrogenation performance of anthraquinone. The experimental results showed that the alloy construction not only reduced the particle size of Pd active component, but also effectively reduced the energy barrier of anthraquinone hydrogenation. More importantly, the Pd—Ir alloy catalyst with higher ordering degree reduced the continuous Pd sites due to more significant geometric effect, which was conducive to prevent the hydrogenation of benzene ring in anthraquinone molecules to generate over-hydrogenation products, and thus improved the selectivity to a certain extent. However, challenges still emerge from the heterogeneity of such a system (namely, the metal atoms can locate at different support sites). Also, owing to high surface free energy, individual metal atoms are generally mobile on the support surface and thus aggregate, especially in reaction processes occurring at elevated temperatures. As a consequence of these points, the development of alternative fabrication strategies to prepare well-defined and stable site-isolated catalysts with higher metal loading are very desirable, albeit challenging.

Trinuclear transition metal-sulfur cluster compound [X₃S₄Lₐ]ⁿ⁺ has attracted extensive attention due to its unsaturated cubane-like structure, stable X—S bond, outstanding stability, relatively independent structures as well as weak agglomeration effect. More importantly, the vertex vacancies in cluster core ions can serve as the capture center to anchor the metal ions (Pd, Pt, Ni, Cu, Ga, etc.) with valence electron number less than 10 to form a complete cubane-like structure, which provides a new platform for the preparation of site-isolated supported metal catalysts.

The present invention utilizes the structural characteristic of unsaturated cubane-like clusters to introduce metal M cations with hydrogenation activity into cluster units to prepare heteronuclear M-based atomic clusters, forming precursors with effectively isolated potential active sites. Then, large-pore Al₂O₃ is used as the support to coordinately load by the temperature-controlled impregnation strategy, and further the treating atmosphere is utilized to induce the formation of sulfur vacancies and promot the change of electronic structure of active M species, and thus construct a highly dispersed supported X₃MSₓ/Al₂O₃ catalyst with Mᵢₛₒ-Vₛ synergistic sites to improve the activity, selectivity and stability of selective hydrogenation, dehydrogenation and dechlorination catalysts.

### SUMMARY OF THE INVENTION

The purpose of the present invention is to provide a supported metal catalyst with synergistic sites, a preparation method therefor and an application thereof. The supported catalyst disclosed by the present invention is expressed as MX₃Sₓ/Al₂O₃, wherein x represents sulfur-containing number in the range of 0<x<4; MX₃Sₓ is an active component, with M as an active metal being one of Pd, Pt, Co, Ni, Ru, Ga, Fe, Cu and Ag; X represents a transition metal being one of Mo, W, Re and Ir; Al₂O₃ is a support; and the corresponding loading of M is 0.03˜5.00 wt. %; The structure of the catalyst is characterized in that the MX₃Sₓ (0<x<4) active component is stably dispersed on the Al₂O₃ support, in which Mᵢₛₒ presents a geometric site isolation, and forms a Mᵢₛₒ-Vₛ synergistic sites with the adjacent S vacancies.

Taking trinuclear transition metal-sulfur cluster as a precursor, M with hydrogenation activity is introduced into the precursor cluster core unit. By adopting temperature-controlled impregnation method to load the active component on Al₂O₃, a series of MX₃Sₓ/Al₂O₃ catalysts with Mᵢₛₒ-Vₛ synergistic sites are obtained after liquid phase reduction and atmosphere treatment. The controllable construction of discontinuous M sites is realized based on the cluster structure confinement effect and charge redistribution. While maintaining the advantages of the above geometric structure, the electron structure of metallic Mᵢₛₒ species can be modified by inducing the generation of S vacancies (Vₛ) through atmosphere treatment, and thus obtain the Mᵢₛₒ-Vₛ synergistic sites with controllable electronic structure and type. This catalyst possesses high conversion and selectivity of target product in selective hydrogenation/dehydrogenation/dechlorination reaction, which is easy to recycle and reuse, and exhibits good stability.

A preparation method of the corresponding supported metal catalyst with synergistic sites disclosed by the present invention comprises the following specific steps:
A. Mix a soluble metal M salt solution uniformly with a cluster compound [X₃S₄(H₂O)_{y}]Cl_{z} solution, wherein the molar ratio of M to X₃ is 4˜25/1, and the concentration of the M salt is 0.0035-0.0171 mol/L;
The corresponding soluble metal M salt is one of Na₂PdCl₄, Pd(NO₃)₂, Pd(C₅H₇O₂)₂, H₂PtCl₆, Pt(NO₃)₂, CoCl₂, Ni(NO₃)₂·6H₂O, NiCl₂, RuCl₃, Ga(NO₃)₃, Fe(NO₃)₃·9H₂O, CuCl₂·2H₂O, Cu(NO₃)₂ and AgNO₃.

The corresponding [X₃S₄(H₂O)_{y}]Cl_{z} is a trinuclear transition metal cluster with an unsaturated cubane structure, wherein X is one of Mo, W, Re and Ir; y=9; when X is Re⁽⁺³⁾, z=1; when X is one of W, Mo and Ir⁽⁺⁴⁾, z=4.
B. Disperse Al₂O₃ support uniformly into the mixed solution in step A, stir at the temperature of 25˜60° C. and the rotation speed of 200˜500 rpm for 2˜6 h until it becomes sticky, and dry it in a constant temperature drying oven at 40˜80° C. for 8˜24 h to obtain [X₃MS₄(H₂O)_{y+1}]^{z+}/Al₂O₃ solid powder;

The corresponding Al₂O₃ support possesses rich pore structure while the crystalline phase is γ or δ, the specific surface area is 70˜190 m²/g, the pore volume is 0.3˜1.3 cm³/g, and the pore size is 15˜30 nm; the theoretical loading of the soluble metal M salt is 0.03˜5.00 wt. %, preferably 0.05˜2.50 wt. %;
C. Add 1.5 g of the solid powder obtained in step B into 5˜30 mL of deionized water, add excessive soluble reducing agent to reduce M²⁺/M³⁺ to M metal, wherein the molar ratio of the reducing agent to the M salt is 3:1˜7:1, stir for 18˜60 min to obtain black suspension, centrifugally wash it to neutrality, and dry it in a constant temperature drying oven at 60° C. for 8˜16 h to obtain X₃MS₄/Al₂O₃;

The corresponding soluble reducing agent is one of NaBH₄, LiBH₄, ascorbic acid and oxalic acid;
D. Place the X₃MS₄/Al₂O₃ obtained in step C into an atmosphere furnace, and heat it to 300˜600° C. at a rate of 5˜20° C./min for 0.25˜6 h of treatment to obtain X₃MSₓ/Al₂O₃ catalyst (0<x<4);

The corresponding treatment atmosphere is one of air, 10 vol. % O₂/N₂, 5˜40 vol. % H₂/N₂ or 5˜40 vol. % CO/N₂; and the corresponding X₃MSₓ/Al₂O₃ catalyst possesses Mᵢₛₒ-Vₛ synergistic sites.

A [X₃S₄(H₂O)_{y}]Cl_{z} cluster solution is prepared by dissolving thiometalate in deionized water to prepare a solution with a concentration of 50˜120 mmol/L, alternately dropping excessive reducing agent and acid solution, and fully stirring at room temperature to obtain a suspension, crystallizing it at 60˜100° C. for 10˜20 h, adding acid solution dropwise to keep pH value of the suspension in the range of 1˜3 during the crystallization process, and making it naturally cool down to room temperature after the color of the suspension turns dark green to obtain a [X₃S₄(H₂O)_{y}]Cl_{z} cluster solution;

The corresponding thiometalate is one of (NH₄)₂MoS₄, (NH₄)₂WS₄, Na₂[Mo₂O₂S₂(cys)₂]·4H₂O, [PPh₄]2[ReS(S₂C₂—(SiMe₃)₂)(μ-S)₂ReS(S₂C₂—(SiMe₃)₂)] and [CpIr(SH)(μ₂-SH)₂IrCp (SH)];

The corresponding soluble reducing agent is one of NaBH₄, LiBH₄, ascorbic acid and oxalic acid;

The corresponding acid solution is one of 6 mol/L HCl solution, 2 mol/L HCl solution and perchloric acid solution.

The preparation method is characterized in that by utilizing the unsaturated cubane-like structure of the trinuclear transition metal-sulfur cluster, M cation with catalytic activity is introduced into the cluster core unit. By using the vertex vacancy as the capturing center, and adjusting the impregnation temperature to maximize the loading of the cluster precursor, as well as depending on the electrostatic adsorption of the support and the confinement of the cluster structural unit, the number of S vacancies and the distance between S vacancies and Mᵢₛₒ sites are effectively controlled through liquid phase reduction and atmosphere treatment at room temperature to obtain supported X₃MSₓ/Al₂O₃ catalyst with Mᵢₛₒ-Vₛ synergistic sites. The preparation method solves the problem that active metal particles with small particle sizes prepared by traditional methods are easy to agglomerate, which guarantees high hydrogenation activity, product selectivity and excellent stability. The preparation method features simple process without the need of adding surfactant. The catalyst is mainly used in selective hydrogenation and dechlorination in important fine chemical processes as well as propane dehydrogenation reaction in petrochemical industry, showing excellent activity and target product selectivity.

FIG. 1 shows X-Ray Diffraction (XRD) results of the Pd^{δ+}Mo₃S₃/Al₂O₃ catalyst prepared in Embodiment 1, from which it can be seen that the prepared catalyst has an outstanding crystalline structure.

FIG. 2 shows CO-IR spectra of the Pd^{δ+}Mo₃S₃/Al₂O₃ catalyst prepared in Embodiment 1. It can be seen that the linear adsorption peak of CO on palladium species appears above 2100 cm⁻¹, but there is no obvious CO bridging-adsorbed peak on Pd⁰, which indicates that the active Pd components in the catalyst exhibits no obvious agglomeration, while continuous Pd sites are effectively isolated.

FIG. 3 shows XPS spectra of the Pd^{δ+}Mo₃S₃/Al₂O₃ catalyst and Pd/Al₂O₃ catalyst prepared in Embodiment 1, where A is the Pd 3d XPS spectra of the Pd^{δ+}Mo₃S₃/Al₂O₃ catalyst and Pd/Al₂O₃ catalyst, while B is the S 2p XPS spectrum of the Pd^{δ+}Mo₃S₃/Al₂O₃ catalyst. It can be seen that the Pd site is electron-deficient, forming Pd^{δ+} species. Compared with Pd/Al₂O₃ catalyst, the electron cloud density of Pd species in Pd^{δ+}Mo₃S₃/Al₂O₃ catalyst is reduced by using molybdenum-sulfur cluster as a precursor. After atmosphere treatment, there is no obvious signal of S species on the surface of the catalyst, indicating that the formation of S vacancy (Vₛ).

FIG. 4 shows the ESR spectra of the Pd^{δ+}Mo₃S₃/Al₂O₃ catalyst and pure Al₂O₃ prepared in Embodiment 1. It can be seen that an obvious S vacancy (g=2.003) is formed, which is related to the results in FIG. 3, indicating the existence of Pd^{δ+}—Vₛ synergistic sites.

FIG. 5 shows the curve of hydrogenation efficiency versus time for the Pd^{δ+}Mo₃Sₓ/Al₂O₃ catalyst prepared in Embodiment 1 in the selective hydrogenation of anthraquinone. When the reaction time is 120 min, the hydrogenation efficiency reaches 15.7 g/L.

FIG. 6 shows the reusability of Pd^{δ+}Mo₃S₃/Al₂O₃ catalyst prepared in Embodiment 1 in selective hydrogenation reaction of anthraquinone. After the catalyst is reused for five times, the hydrogenation efficiency remains at 11.2 g/L and the target product selectivity is 96%±2%.

FIG. 7 shows the catalytic results of the PtMo₃S₃/Al₂O₃ catalyst prepared in Embodiment 2 in propane dehydrogenation, where A is the curve of propane conversion versus time, and B is the curve of propylene selectivity versus time. When the reaction temperature is 580° C., the initial conversion is 68% and the propylene selectivity is 90%.

FIG. 8 shows the catalytic results of NiMo₃S_{2.1}/Al₂O₃ catalyst prepared in Embodiment 3 in the hydrodechlorination of dichloroethane, where A is the histogram of conversion and selectivity, and B is the reusability curve. When the reaction temperature is 300° C., the initial conversion is 85% and the olefin selectivity is 96%.

### THE BENEFICIAL EFFECTS OF THE INVENTION

Based on the structural characteristics of trinuclear transition metal-sulfur cluster, a novel MX₃Sₓ/Al₂O₃ (0<x<4) catalyst with Mᵢₛₒ-Vₛ synergistic sites is obtained by utilizing the unsaturated cubane-like structure of the trinuclear transition metal-sulfur cluster, M cation with catalytic activity is introduced into the cluster core unit. By using the vertex vacancy as the capturing center, and adjusting the impregnation temperature to maximize the loading of the cluster precursor, as well as depending on the electrostatic adsorption of the support and the confinement of the cluster structural unit. The preparation method features simple process without the need of adding surfactant.

The prepared active metal components are uniformly dispersed on the surface of the support, which realizes the effective isolation of continuous M sites. By effectively controlling the number of S vacancies and the distance between S vacancies and Mᵢₛₒ sites, X₃MSₓ/Al₂O₃ catalyst with Mᵢₛₒ-Vₛ sites with enhanced synergistic effect is obtained. The catalyst can be applied to selective hydrogenation of C═O bond, hydrodechlorination of C—Cl bond and dehydrogenation of C—H bond, and exhibits excellent activity and selectivity, easy recovery and reuse, and good stability on the premise of reducing the amount of precious metals.

### BRIEF DESCRIPTION OF THE DRAWINGS

FIG. 1 shows XRD results of the Pd^{δ+}Mo₃S₃/Al₂O₃ catalyst prepared in Embodiment 1.

FIG. 2 shows CO-IR spectra of the Pd^{δ+}Mo₃S₃/Al₂O₃ catalyst prepared in Embodiment 1.

FIG. 3 shows the XPS spectra of the Pd^{δ+}Mo₃S₃/Al₂O₃ catalyst and Pd/Al₂O₃ catalyst prepared in Embodiment 1, where A is the Pd 3d XPS spectra of the Pd^{δ+}Mo₃S₃/Al₂O₃ catalyst and Pd/Al₂O₃ catalyst, while B is the S 2p XPS spectrum of the Pd^{δ+}Mo₃S₃/Al₂O₃ catalyst.

FIG. 4 shows the ESR spectra of the Pd^{δ+}Mo₃S₃/Al₂O₃ catalyst and pure Al₂O₃ prepared in Embodiment 1.

FIG. 5 shows the curve of hydrogenation efficiency versus time of the Pd^{δ+}Mo₃S₃/Al₂O₃ catalyst prepared in Embodiment 1 in the selective hydrogenation of anthraquinone.

FIG. 6 shows the reusability of the Pd^{δ+}Mo₃S₃/Al₂O₃ catalyst prepared in Embodiment 1 in selective hydrogenation reaction of anthraquinone.

FIG. 7 shows the catalytic results of the PtMo₃S₃/Al₂O₃ catalyst prepared in Embodiment 2 in propane dehydrogenation, where A is the curve of propane conversion versus time, and B is the curve of propylene selectivity versus time.

FIG. 8 shows the catalytic results of NiMo₃S_{2.1}/Al₂O₃ catalyst prepared in Embodiment 3 in the hydrodechlorination of dichloroethane, where A is the histogram of conversion and selectivity, and B is the reusability curve.

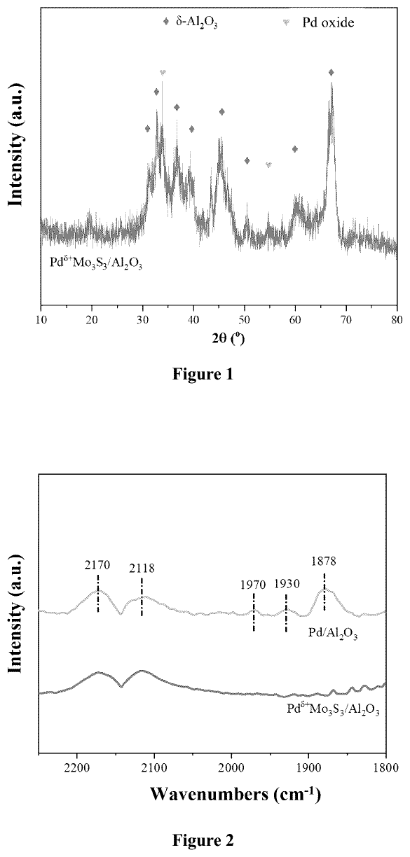

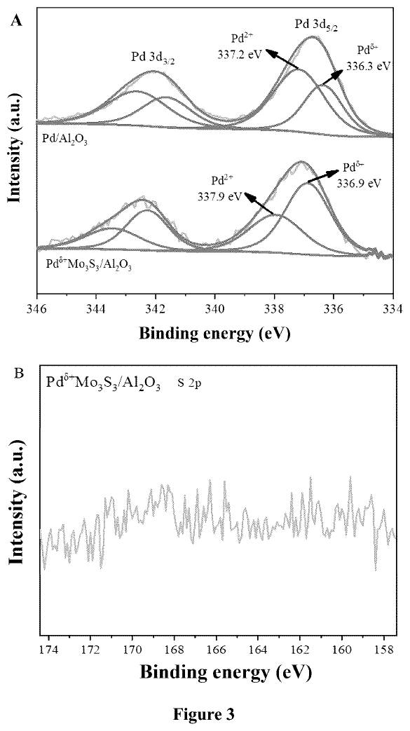

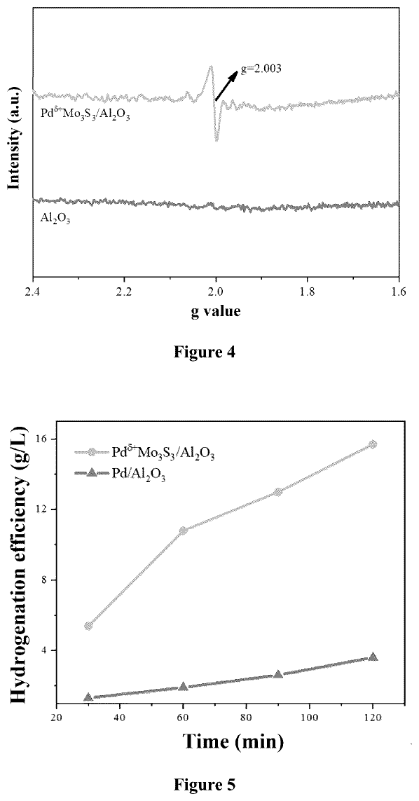

### DETAILED DESCRIPTION OF EMBODIMENTS

### Embodiment 1

A. Stir and mix 5.64 mL soluble metallic Na₂PdCl₄ solution with a concentration of 50 mmol/L and 5 mL [Mo₃S₄(H₂O)₉]Cl₄ solution with a concentration of 12.8 mmol/L to obtain a solution containing heteronuclear Pd-based [Mo₃PdS₄(H₂O)₁₀]Cl₄ atomic cluster;
B. Calcine pseudo-boehmite at 960° C. to obtain Al₂O₃ support with rich pore structure. According to the theoretical loading of the metallic M being 0.4 wt. %, disperse 1.5 g Al₂O₃ support uniformly into 10.64 mL mixed solution obtained in step A, continuously stir at 60° C. and 300 rpm for 4 h until it becomes sticky, and dry it in a constant temperature drying oven at 60° C. for 12 h to obtain [Mo₃PdS₄(H₂O)₁₀]Cl₄/Al₂O₃ solid powder;
C. Add 1.5 g solid powder obtained in step B into 10 mL deionized water, add 0.0533 g NaBH₄ as a reducing agent, stir for 30 minutes, centrifuge and wash the obtained black suspension to neutral while washing away soluble sodium salts, and dry at 60° C. in a constant temperature drying oven for 12 h to obtain Mo₃PdS₄/Al₂O₃;
D. Place the Mo₃PdS₄/Al₂O₃ obtained in step C into an atmosphere furnace, heat it to 450° C. at the rate of 10° C./min in air and calcine it for 4 h to obtain the Mo₃PdSₓ/Al₂O₃ catalyst; and the corresponding Mo₃PdSₓ/Al₂O₃ catalyst (x=3) has Pdᵢₛₒ—Vₛ synergistic sites.

It can be seen from Table 1 that the catalyst has rich pore structure and large specific surface area.

TABLE 1
Test results of specific surface area and
pore size of Al₂O₃ and PdMo₃S₃/Al₂O₃

| | Specific | Specific | |
|---|---|---|---|
| | Surface Area | Pore Volume | Pore Size |
| Sample | (m²/g) | (cm³/g) | (nm) |
| Al₂O₃ | 87 | 0.37 | 16.69 |
| Pd^{δ+}Mo₃S₃/Al₂O₃ | 86 | 0.34 | 15.55 |

The corresponding prepared catalyst is used in the selective hydrogenation experiment of anthraquinone:

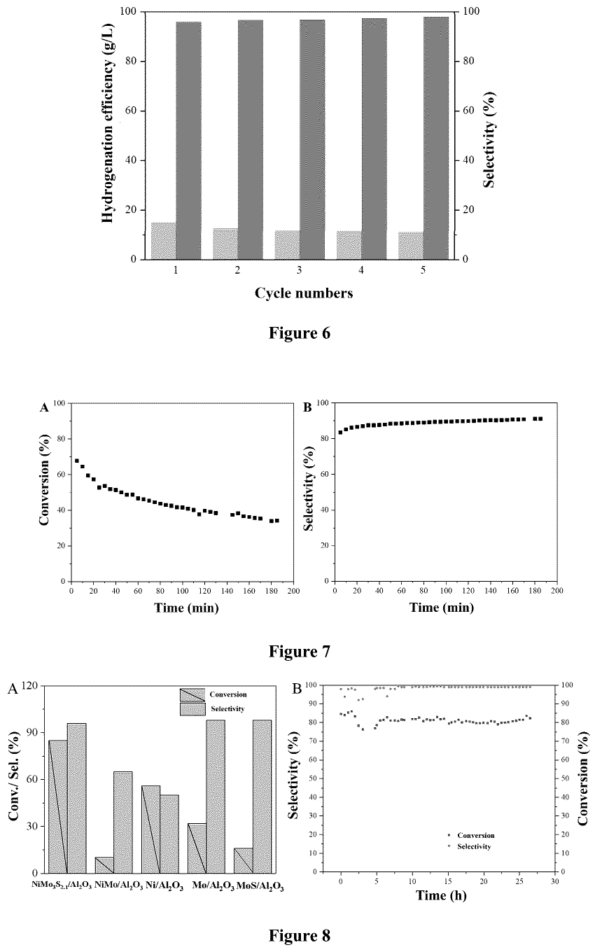

Weigh 25 mg of the corresponding catalyst and 60 mL 2-ethylanthraquinone working solution (120 g/L) and put them into a 100 mL reactor. Introduce hydrogen to 0.3 MPa, heat it to 50° C., and then turn on stirring (at a speed of 1,000 rpm), and periodically open the liquid valve to take 1 mL working solution for analysis. See the results in FIG. 5 and FIG. 6.

It can be seen from FIG. 5 that the hydrogenation efficiency of the catalyst reaches 15.7 g/L when the reaction time is 120 min. Compared with the 11.9 g/L in the Enhanced Catalytic Performance of Pd—Ga Bimetallic Catalysts for 2-ethylanthraquinone Hydrogenation. Appl Organometal Chem. 2019, 33, e5076, the hydrogenation efficiency is significantly improved. It can be seen from FIG. 6 that the hydrogenation efficiency of the catalyst remains at 11.2 g/L and the target product selectivity is 96% 2% after reusing for five times.

### Embodiment 2

A. Stir and mix 10.0 mL soluble metallic H₂PtCl₆ solution with a concentration of 15.0 mmol/L and [Mo₃S₄(H₂O)₉]Cl₄ solution at a Pt/Mo₃S₄ molar ratio of 1:1 to obtain a solution containing heteronuclear Pt-based [Mo₃PtS₄(H₂O)₁₀]Cl₄ atomic cluster;
B. According to the theoretical loading of the metallic M being 2.00 wt. %, disperse 1.48 g Al₂O₃ support uniformly into the mixed solution obtained in step A, continuously stir at 60° C. and 300 rpm for 4 h until it becomes sticky, and dry it in a constant temperature drying oven at 60° C. for 24 h to obtain [Mo₃PtS₄(H₂O)₁₀]Cl₄/Al₂O₃ solid powder;
C. Add 1.5 g solid powder obtained in step B into 10 mL deionized water, add 0.0533 g NaBH₄ as a reducing agent, stir for 30 minutes, centrifuge and wash the obtained black suspension to neutral while washing away soluble sodium salts, and dry it at 60° C. in a constant temperature drying oven for 12 h to obtain Mo₃PdS₄/Al₂O₃;
D. Place the Mo₃PdS₄/Al₂O₃ obtained in step C into an atmosphere furnace, heat it to 570° C. at the rate of 10° C./min in 10% H₂/N₂ and treat it for 0.33 h to obtain the Mo₃PtSₓ/Al₂O₃ catalyst; and the corresponding Mo₃PtSₓ/Al₂O₃ catalyst (x=3) has Ptᵢₛₒ-Vₛ synergistic sites.

The catalyst is used in propane dehydrogenation:

Weigh 0.20 g catalyst and mix it with 1.80 g quartz sand with a particle size of 40-70 mesh thoroughly, and then load it into a quartz tube reactor with a diameter of 8 mm. The reaction feed gas is composed of 2.0% propane, 4% hydrogen and 94% nitrogen equilibrium gas, and the testing temperature is 580° C. The gas chromatography is used to analyze the composition and content of reactants and products. When the reactor reaches the specified temperature, records are made every 5 minutes. The results of propane conversion rate and selectivity of the catalyst at 580° C. are shown in Table 2.

TABLE 2
Catalytic performance

Embodiment 1

| Sample | Selectivity (%) | Conversion rate (%) |
|---|---|---|
| Embodiment 2 | 90 | 68 |

The Pt—Sn catalyst in the literature ACS Catal. 2021, 11, 8, 4401-4410 is a commonly used catalyst for industrial propane dehydrogenation, with a selectivity of 92% and a conversion rate of 40% for propane dehydrogenation at 600° C. It can be seen from Table 2 that compared with the Pt—Sn catalyst reported in the literature, the PtMo₃S₃/Al₂O₃ catalyst prepared by the present invention has similar selectivity and more preferred activity at a lower temperature.

### Embodiment 3

A. Stir and mix 6.7 mL soluble metallic Ni(NO₃)₂·6H₂O solution with a concentration of 0.19 mol/L and 5 mL [Mo₃S₄(H₂O)₉]Cl₄ solution with a concentration of 12.8 mmol/L to obtain a solution containing heteronuclear Ni-based [Mo₃NiS₄(H₂O)₁₀]Cl₄ atomic cluster;
B. According to the theoretical loading of metallic Ni being 2.5 wt. % of the catalyst, disperse 3.0 g Al₂O₃ support uniformly into 10.00 mL mixed solution obtained in step A, continuously stir at 60° C. and 400 rpm for 5 h until it becomes sticky, and dry it in a constant temperature drying oven at 60° C. for 12 h to obtain [Mo₃NiS₄(H₂O)₁₀]Cl₄/Al₂O₃ solid powder;
C. Add 1.5 g solid powder obtained in step B into 10 mL deionized water, add 0.0533 g NaBH₄ as a reducing agent, stir for 30 minutes, centrifuge and wash the obtained black suspension to neutral while washing away soluble sodium salts, and dry it at 60° C. in a constant temperature drying oven for 12 h to obtain Mo₃NiS₄/Al₂O₃;
D. Place the Mo₃NiS₄/Al₂O₃ obtained in step C into an atmosphere furnace, heat it to 450° C. at the rate of 10° C./min in 10 vol. % H₂/N₂ and conduct heat treatment for 3 h to obtain the Mo₃NiSₓ/Al₂O₃ catalyst; and the corresponding Mo₃NiSₓ/Al₂O₃ catalyst (x=2.1) has Niᵢₛₒ—Vₛ synergistic sites.

The catalyst is used in the 1,2-dichloroethane hydrodechlorination:

Weigh 0.30 g catalyst and mix it with 1.40 g quartz sand with a particle size of 40˜70 mesh thoroughly, and then load it into a quartz tube reactor with a diameter of 7 mm. The reaction feed gas is composed of 92% 1,2-dichloroethane mixture and 8% hydrogen/nitrogen mixture, and the testing temperature is 300° C. The gas chromatography is used to analyze the composition and content of reactants and products. When the reactor reaches the specified temperature, records are made every 10 minutes. The results of 1,2-dichloroethane conversion rate and selectivity of the catalyst at 300° C. are shown in Table 3.

TABLE 3
Catalytic performance

Embodiment 1

| Sample | Selectivity (%) | Conversion rate (%) |
|---|---|---|
| Embodiment 3 | 96 | 85 |

According to the literature Chem. Commun. 2020, 56, 6985, the selectivity of 17Ni-PC@SBA-15 catalyst for the hydrodechlorination of 1,2-dichloroethane at 300° C. is 90%, and the conversion is 65.5%. It can be seen from Table 3 that compared with the 17Ni-PC@SBA-15 catalyst reported in the literature, the selectivity and activity of the NiMo₃S_{2.1}/Al₂O₃ catalyst prepared by the present invention are higher than those of the above-mentioned 17Ni-PC@SBA-15 catalyst at the same temperature.

### Embodiment 4

A. Stir and mix 5.64 mL soluble metallic Na₂PdCl₄ solution with a concentration of 50 mmol/L and 5 mL [Re₃S₄(H₂O)₉]Cl solution with a concentration of 12.8 mmol/L to obtain a solution containing heteronuclear Pd-based [Re₃PdS₄(H₂O)₉]Cl atomic cluster;
B. Calcine pseudo-boehmite at 960° C. to obtain Al₂O₃ support with rich pore structure. Disperse 1.5 g Al₂O₃ support into 10.64 mL mixed solution obtained in step A at room temperature, with the loaded active metal Pd being 0.4 wt. %. Stir the mixture continuously for 4 h at 60° C. and 300 rpm until it becomes sticky, and dry it in a constant temperature drying oven at 60° C. for 12 h to obtain [Re₃PdS₄(H₂O)₉]Cl/Al₂O₃ solid powder.
C. Add 1.5 g solid powder obtained in step B into 10 mL deionized water, add 0.0533 g NaBH₄ as a reducing agent, stir for 30 minutes, centrifuge and wash the obtained black suspension to neutral while washing away soluble sodium salts, and have it dried at 60° C. in a constant temperature drying oven for 12 h to obtain Re₃PdS₄/Al₂O₃;
D. Place the Re₃PdS₄/Al₂O₃ obtained in step C into an atmosphere furnace, heat it to 450° C. at the rate of 10° C./min in air and calcine it for 4 h to obtain the Re₃PdSₓ/Al₂O₃ catalyst; and the corresponding Re₃PdSₓ/Al₂O₃ catalyst (x=3) has Pdᵢₛₒ—Vₛ synergistic sites.

### Embodiment 5

A. Stir and mix 14.10 mL soluble metallic Na₂PdCl₄ salt solution with a concentration of 50 mmol/L and 5 ml [Mo₃S₄(H₂O)₉]Cl₄ solution with a concentration of 12.8 mmol/L to obtain a solution containing heteronuclear Pd-based [Mo₃PdS₄(H₂O)₁₀] atomic cluster;
B. Calcine pseudo-boehmite at 960° C. to obtain Al₂O₃ support with rich pore structure. Disperse 1.5 g Al₂O₃ support into 12.05 mL mixed solution obtained in step A at room temperature, in which the supported active metal Pd is 1.0 wt. %. Stir the mixture continuously for 4 h at 40° C. and 300 rpm until it becomes sticky, and dry it in a constant temperature drying oven at 40° C. for 12 h to obtain [Mo₃PdS₄(H₂O)₁₀]Cl₄/Al₂O₃ solid powder.
C. Add 1.5 g solid powder obtained in step B into 10 mL deionized water, add 0.0666 g NaBH₄ as a reducing agent, stir for 30 minutes, centrifuge and wash the obtained black suspension to neutral while washing away soluble sodium salts, and dry it at 60° C. in a constant temperature drying oven for 12 h to obtain Mo₃PdS₄/Al₂O₃;
D. Place the Mo₃PdS₄/Al₂O₃ obtained in step C into an atmosphere furnace, heat it to 450° C. at the rate of 10° C./min in air and calcine it for 4 h to obtain the Mo₃PdSₓ/Al₂O₃ catalyst; and the corresponding Mo₃PdSₓ/Al₂O₃ catalyst (x=3) has Pdᵢₛₒ—Vₛ synergistic sites.

### Embodiment 6

A. Stir and mix 6.7 mL soluble metallic CuCl₂·2H₂O solution with a concentration of 0.19 mol/L and 5 mL [Mo₃S₄(H₂O)₉]Cl₄ solution with a concentration of 12.8 mmol/L to obtain a solution containing heteronuclear Cu-based [Mo₃CuS₄(H₂O)₁₀]Cl atomic cluster;
B. According to the theoretical loading of metallic Cu being 2.50 wt. %, disperse 3.0 g Al₂O₃ support uniformly into 10.00 mL mixed solution obtained in step A, continuously stir at 60° C. and 400 rpm for 5 h until it becomes sticky, and dry it in a constant temperature drying oven at 60° C. for 12 h to obtain [Mo₃CuS₄(H₂O)₁₀]Cl₄/Al₂O₃ solid powder;
C. Add 1.5 g solid powder obtained in step B into 10 mL deionized water, add 0.0533 g NaBH₄ as a reducing agent, stir for 30 minutes, centrifuge and wash the obtained black suspension to neutral while washing away soluble sodium salts, and dry it at 60° C. in a constant temperature drying oven for 12 h to obtain Mo₃CuS₄/Al₂O₃;
D. Place the Mo₃CuS₄/Al₂O₃ obtained in step C into an atmosphere furnace, heat it to 400° C. at the rate of 5° C./min in 10 vol. % H₂/N₂ and conduct heat treatment for 3 h to obtain the Mo₃CuSₓ/Al₂O₃ catalyst; and the corresponding Mo₃CuSₓ/Al₂O₃ catalyst (x=2.5) has Cuᵢₛₒ—Vₛ synergistic sites.

1. A preparation method of a supported metal catalyst with synergistic sites, characterized in that the preparation method comprises the following steps:
A. Mix a soluble metal M salt solution uniformly with a solution of [X3S4(H2O)y]Clz cluster compound to obtain a [X3MS4(H2O)y+1]z+ solution;
B. Disperse Al2O3 support uniformly into the solution obtained in step A, stir at the temperature of 25˜60° C. and the rotation speed of 200˜500 rpm for 2˜6 h until it becomes sticky, and dry it in a constant temperature drying oven at 40˜80° C. for 8˜24 h to obtain [X3MS4(H2O)y+1]z+/Al2O3 solid powder;
C. Add 1.5 g solid powder obtained in step B into 5˜30 mL of deionized water, add excessive soluble reducing agent to reduce M2+/M3+ to metal M, wherein the molar ratio of the reducing agent to the M salt is 3:1˜7:1, stir for 18˜60 min to obtain black suspension, centrifugally wash it to neutrality, and dry it in a constant temperature drying oven at 60° C. for 8˜16 h to obtain X3MS4/Al2O3;
D. Place the X3MS4/Al2O3 obtained in step C into an atmosphere furnace, and heat it to 300˜600° C. at a rate of 5˜20° C./min for 0.25˜6 h of treatment to obtain X3MSx/Al2O3 catalyst (0<x<4);
The corresponding treatment atmosphere is one of air, 10 vol. % O2/N2, 5˜40 vol. % H2/N2 or 5˜40 vol. % CO/N2; The corresponding X3MSx/Al2O3 catalyst possesses Miso-Vs synergistic sites.

2. A preparation method of a supported catalyst with synergistic sites according to claim 1, characterized in that the corresponding [X3S4(H2O)y]Clz in step A is a trinuclear transition metal-sulfur cluster compound with an unsaturated cubane structure, wherein X is one of Mo, W, Re and Ir; y=9; when X is Re (+3), z=1; when X is one of W, Mo and Ir(+4), z=4.

3. A preparation method of a supported catalyst with synergistic sites according to claim 1, characterized in that the corresponding soluble metal M salt in step A is one of Na2PdCl4, Pd(NO3)2, Pd(C5H7O2)2, H2PtCl6, Pt(NO3)2, CoCl2, Ni(NO3)2·6H2O, NiCl2, RuCl3, Ga(NO3)3, Fe(NO3)3·9H2O, CuCl2·2H2O, Cu(NO3)2 and AgNO3.

4. A preparation method of a supported catalyst with synergistic sites according to claim 1, characterized in that the molar ratio of M to X3 in step A is 4˜25/1, and the concentration of the M salt is 0.0035˜0.0171 mol/L.

5. A preparation method of a supported catalyst with synergistic sites according to claim 1, characterized in that the theoretical loading of the soluble metal M salt in step B is 0.03˜5.00 wt. % of the catalyst, preferably 0.05˜2.50 wt. %; the corresponding Al2O3 support possesses rich pore structure while the crystalline phases is γ or δ, the specific surface area is 70˜190 m2/g, the pore volume is 0.3˜1.3 cm3/g, and the pore size is 15˜30 nm; the soluble reducing agent in step C is one of NaBH4, LiBH4, ascorbic acid or oxalic acid.

6. A supported catalyst with synergistic sites according to claim 1, characterized in that the preparation method of a [X3 S4(H2O)y]Clz cluster solution comprises the following steps: Dissolve thiometalate in deionized water to prepare a solution with a concentration of 50˜120 mmol/L, alternately drop excessive reducing agent and acid solution, and fully stir at room temperature to obtain a suspension, crystallize it at 60˜100° C. for 10˜20 h, add acid solution dropwise to keep pH value of the suspension in the range of 1˜3 during the crystallization process, and make it naturally cool down to room temperature after the color of the suspension turns dark green to obtain a [X3S4(H2O)y]Clz cluster solution.

7. A supported catalyst with synergistic sites prepared by the method according to claim 1, characterized in that the catalyst is expressed as MX3Sx/Al2O3, wherein x represents sulfur-containing number in the range of 0<x<4; MX3Sx is an active component, with M as an active metal being one of Pd, Pt, Co, Ni, Ru, Ga, Fe, Cu and Ag; X represents a transition metal being one of Mo, W, Re and Ir; Al2O3 is a support; and the corresponding loading of M is 0.03˜5.00 wt. %; The structure of the catalyst is characterized in that the MX3Sx (0<x<4) active component is stably dispersed on the Al2O3 support, in which Miso presents a geometric site isolation, and forms a Miso-Vs synergistic sites with the adjacent S vacancies.

8. An application of a supported catalyst with synergistic sites prepared by the method according to claim 7 to catalyze the selective hydrogenation of unsaturated carbon-oxygen bonds, characterized in that the corresponding catalyst and 2-ethylanthraquinone working solution are weighed and loaded into a reactor, hydrogen is introduced into the reactor until the pressure reaches 0.3 MPa, the stirring is turned on at a rotation speed of 1000 rpm until the reactor is heated to 50° C., wherein the amount of the catalyst is 0.02˜0.10 g, the reaction temperature is 40˜80° C., the rotation speed is 700˜1000 rpm, the 2-ethylanthraquinone working solution is 100˜150 g/L, and the test pressure is 1˜4 bar. The liquid phase valve is opened every 30 minutes to take 1 mL working solution and mix it with 20 mL deionized water in a 250 mL separatory funnel and oxidize it with pure oxygen. When the upper organic phase turns from brownish black to bright yellow, stop introducing oxygen, and extract with deionized water for three times. The lower water phase obtained is titrated with 0.02 mol/L potassium permanganate solution under acidic conditions. Finally, the concentration of hydrogen peroxide is calculated.

9. An application of a supported catalyst with synergistic sites prepared by the method according to claim 7 to catalyze alkanes to produce olefins, characterized in that the catalyst is weighed and fully mixed with quartz sand possessing a particle size of 40˜70 meshes, and then loaded into a quartz tube reactor with a diameter of 8 mm, the feed gas composed of 2.0% propane/4% hydrogen/94% nitrogen equilibrium gas is introduced, wherein the molar ratio of hydrogen to alkane is 1/1˜2/1. The catalytic performance is tested under the reaction temperature of 550˜580° C., pressure of 1˜4 bar and space velocity of 3000˜6000 h˜1. The composition and content of reactants and products are analyzed by gas chromatography with recording every 10 minutes.

10. An application of a supported catalyst with synergistic sites prepared by the method according to claim 7 to catalyze the selective hydrodechlorination of chlorine-containing organic compounds, characterized in that the catalyst is weighed and fully mixed with quartz sand possessing a particle size of 40˜70 meshes, and then loaded into a quartz tube reactor with a diameter of 7 mm, the feed gas composed of 0.14% dichloroethane/0.8% hydrogen/99.06% nitrogen equilibrium gas is introduced. A catalytic reaction is conducted under the certain conditions, wherein the amount of the catalyst is 0.02˜0.50 g, the reaction temperature is 170˜500° C., the ratio of hydrogen to chlorine is 1/1˜10/1, and the space velocity is 2000˜12000 h˜1. The composition and content of reactants and products are analyzed by gas chromatography with recording every 10 minutes.
